(19) United States
(12) Patent Application Publication
(10) Pub. No.: US 2023/0181067 A1
(43) Pub. Date: Jun. 15, 2023

(54) IN-CELL OPTICAL BIOMETRICS SENSOR

(71) Applicant: Egis Technology lnc., Hsinchu City (TW)

(72) Inventors: CHEN-CHIH FAN, Hsinchu City (TW); CHENCHANG HUANG, Hsinchu City (TW); BRUCE C.S. CHOU, Hsinchu City (TW)

(21) Appl. No.: 17/912,441

(22) PCT Filed: Sep. 11, 2020
(86) PCT No.: PCT/CN2020/114731

§ 371 Date: Sep. 16, 2022

Related U.S. Application Data
(60) Provisional application No. 63/005,703, filed on Apr. 6, 2020.
(60) Provisional application No. 63/010,931, filed on Apr. 16, 2020.

(51) Int. Cl. A61B 5/1455; A61B 5/1171

(52) U.S. Cl. CPC A61B 5/1455; A61B 5/1176

(57) ABSTRACT

An in-cell optical biometrics sensor includes: a display unit sets each including one or multiple display units; optical sensing cells respectively disposed in gaps between the display unit sets; and optical modules respectively disposed adjacently to the optical sensing cells, wherein each optical module includes a light shielding layer for shielding stray light, and the optical sensing cells sense biometrics characteristics of an object through the optical modules. Thus, the optical biometrics sensor can be integrated in a display panel to provide partial or full-display optical biometrics characteristics sensing functions.

### CROSS-REFERENCE TO RELATED APPLICATIONS

This application is a U.S. National Stage under 35 U.S.C § 371 of International Application No. PCT/CN2020/114731 filed on Sep. 11, 2020, which claims priorities of U.S. Provisional Application No. 63/005,703 filed on Apr. 6, 2020, and U.S. Provisional Application No. 63/010,931 filed on Apr. 16, 2020 under 35 U.S.C. § 119(e), the entire contents of which are hereby incorporated by reference.

### BACKGROUND OF THE INVENTION

### Field of the Invention

This disclosure relates to an optical biometrics sensor, and more particularly to an in-cell optical biometrics sensor, wherein the optical biometrics sensor is integrated into a display panel to provide partial or full-display optical biometrics characteristics sensing functions.

### Description of the Related Art

Today's mobile electronic devices (e.g., mobile phones, tablet computers, notebook computers and the like) are usually equipped with user biometrics recognition systems including different techniques relating to, for example, fingerprint, face, iris and the like, to protect security of personal data. Portable devices applied to mobile phones, smart watches and the like also have the mobile payment function, which further becomes a standard function for the user's biometrics recognition. The portable device, such as the mobile phone and the like, is further developed toward the full-display (or super-narrow border) trend, so that conventional capacitive fingerprint buttons can no longer be used, and new minimized optical imaging devices, some of which are very similar to the conventional camera module having complementary metal-oxide semiconductor (CMOS) image sensor (referred to as CIS) sensing members and an optical lens module, are thus evolved. The minimized optical imaging device is disposed under the display as an under-display device. The image of the object (more particularly the fingerprint) placed above the display can be captured through the partial light-transmitting display (more particularly the organic light emitting diode (OLED) display), and this can be called as fingerprint on display (FOD).

However, the FOD technology encounters certain difficulties. Because the light representative of the fingerprint image needs to pass through the display panel, the fingerprint image signal is combined with the light-transmitting pattern of the panel, the signal processing becomes difficult and a complicated image processing method is required to solve this problem. Meanwhile, different display panels have different light-transmitting rates and different light-transmitting patterns, so solutions are always needed. More importantly, with the growing trend of the development of the display panel, the opaque technology may be finally developed. At this time, the under-display optical fingerprint sensing becomes useless. To this end, this disclosure proposes how to design an in-cell optical biometrics sensor in order to solve the above-mentioned problem.

### BRIEF SUMMARY OF THE INVENTION

It is therefore an objective of this disclosure to provide an in-cell optical biometrics sensor, wherein the optical biometrics sensor is integrated into a display panel to provide partial or full-display optical biometrics characteristics sensing functions.

To achieve the above-identified objective, this disclosure provides an in-cell optical biometrics sensor including: display unit sets each including one or multiple display units; optical sensing cells respectively disposed in gaps between the display unit sets; and optical modules respectively disposed adjacently to the optical sensing cells, wherein each of the optical modules includes a light shielding layer for shielding stray light, and the optical sensing cells sense biometrics characteristics of an object through the optical modules.

With the above-mentioned in-cell optical biometrics sensor, sensing cells for sensing biometrics characteristics may be integrated with the display panel to provide a display panel with an in-cell optical biometrics sensor, so that the display function and the biometrics characteristics sensing function can be integrated together, and that the assembling cost, and the positioning structure or bonding structure necessary for assembling can be saved. In addition, because the sensing cell can be configured in conjunction with the display pixel of the display panel, it is possible to design the in-cell optical biometrics sensor having the full-display biometrics characteristics sensing function, so that the displaying of the electronic apparatus and the biometrics characteristics sensing convenience are further enhanced.

In order to make the above-mentioned content of this disclosure more obvious and be easily understood, preferred embodiments will be described in detail as follows in conjunction with the accompanying drawings.

### BRIEF DESCRIPTION OF THE SEVERAL VIEWS OF THE DRAWINGS

FIG. 1 is a schematically partial cross-sectional view showing an in-cell optical biometrics sensor according to a preferred embodiment of this disclosure.

FIGS. 2A to 2C are schematically cross-sectional views showing three examples of an optical module of FIG. 1.

FIGS. 3A to 3C are schematically cross-sectional views showing three examples of the optical module of FIG. 1.

FIGS. 4A to 4D are schematically cross-sectional views showing four examples of the optical module of FIG. 1.

FIGS. 5 to 7 are schematically partial cross-sectional views showing three modified examples of the in-cell optical biometrics sensor of FIG. 1.

FIGS. 8 to 10 are schematically partial cross-sectional views respectively showing modified examples of the in-cell optical biometrics sensor of FIGS. 5 to 7.

### SYMBOLS

F: object

G: gap

L: light

10: display cover layer

11: lower surface

20: display unit set

21, 22, 23: display unit

30: sensing substrate

31: sensing cell

32: optical sensing cell

33: optical module

34: light shielding layer

34A: aperture

35: transparent dielectric layer

36: micro lens

37: OLED substrate

38: TFT layer

39: TFT array substrate

41: second light shielding layer

41A: second aperture

42: third light shielding layer

42A: third aperture

50: lower light shielding layer

80: liquid crystal display material

90: protection cover layer

100: in-cell optical biometrics sensor

### DETAILED DESCRIPTION OF THE INVENTION

This disclosure provides an in-cell optical biometrics sensor, more particularly an in-cell optical fingerprint sensor, in which sensing cells or optical sensing cells and collimating structures required for optical fingerprint sensing are integrated into an OLED display, a thin film transistor (TFT) liquid crystal display (LCD), a micro light emitting diode (μ LED) display or any other future display to implement the partial or full-screen fingerprint sensing application.

In the in-cell optical biometrics sensor of this disclosure, structures applicable to the LCD may also be applied to the OLED display, other existing displays or any other future display, and structures applicable to the OLED (or μ LED) display may also be applied to LCD, other existing displays or any other future display. That is, the micro lens or the collimator may be disposed on an upper substrate or a lower substrate of the display to provide in-cell optical biometrics characteristics sensing for the LCD, OLED display, μ LED display and the like.

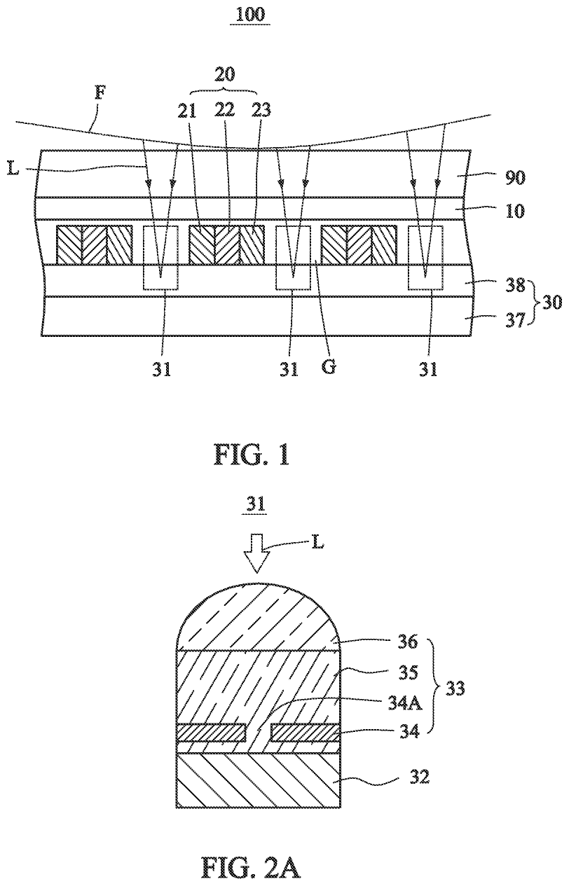

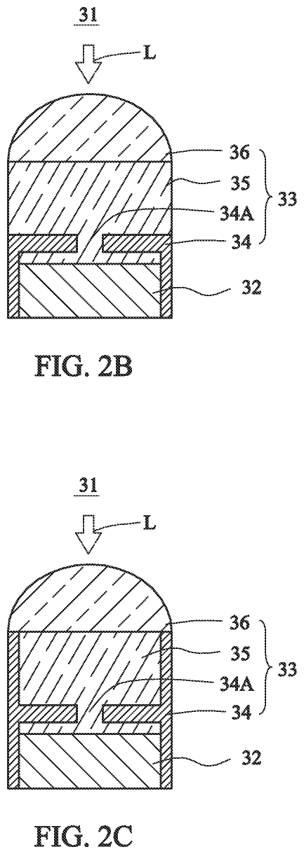

FIG. 1 is a schematically partial cross-sectional view showing an in-cell optical biometrics sensor according to a preferred embodiment of this disclosure. FIGS. 2A to 2C are schematically cross-sectional views showing three examples of an optical module of FIG. 1. Referring to FIGS. 1 and 2A, an in-cell optical biometrics sensor 100 of this embodiment includes display unit sets 20, optical sensing cells 32 and optical modules 33. From another point of view, the in-cell optical biometrics sensor 100 includes a display cover layer 10, the display unit sets 20 and a sensing substrate 30, wherein the optical sensing cells 32 are disposed on the sensing substrate 30.

The display cover layer 10 may be an upper glass substrate, a lower glass substrate, or any other light-transmitting substrate (e.g., a polymeric substrate) of an existing OLED or μ LED display panel. Herein, the upper glass substrate is explained as an example. Of course, it is to be noted that a flexible OLED panel in one example does not have the display cover layer 10.

The display unit set 20 includes one or multiple display units 21 to 23 for displaying information. In this example, the display units 21, 22 and 23 are respectively green, red and blue light-emitting units functioning to display information in the OLED display panel. However, this disclosure is not restricted thereto because it is also applicable to the occasion of the display unit having one single color.

In this embodiment, the sensing substrate 30 includes the optical sensing cells 32 (FIG. 2A). The display unit sets 20 are disposed between the display cover layer 10 and the sensing substrate 30. The optical sensing cells 32 for sensing biometrics characteristics of an object F are respectively disposed in gaps G between the display unit sets 20. Although the sensing substrate 30 including the optical sensing cells 32 is explained as an example, this disclosure is not restricted thereto. As long as the optical sensing cells 32 of this embodiment can be provided, the effect of this embodiment can be achieved. The object F is disposed above the display cover layer 10. It is worth noting that the display cover layer 10 is an optional element. When the display cover layer 10 is omitted, the display unit sets 20 may be disposed on or above the sensing substrate 30. That is, the display unit sets 20 are disposed above the optical sensing cells 32. However, this disclosure is not restricted thereto. The optical sensing cell 32 is, for example, a photodiode, a PIN photodiode, an organic photodiode (OPD) or any non-diode type optical sensing cell structure, and converts optical energy of light L, coming from the object F, into electrical energy. Therefore, an in-cell optical biometrics sensor 100 can be obtained, wherein sensing cells 31 and display pixels including the display unit sets 20 may be integrally manufactured to achieve the displaying function and the biometrics characteristics sensing function. Although the in-cell optical biometrics sensor 100 is explained by taking a fingerprint sensor as an example, this disclosure is not restricted thereto. In other examples, the in-cell optical biometrics sensor 100 may also sense an image of any object, such as biometrics characteristics of the finger including the vein image, blood oxygen concentration image and the like, or biometrics characteristics of the face, iris and the like.

Because the optical sensing cells 32 are disposed in the gaps between the display pixels of the original display, the optical sensing cells 32 may also be configured as a full-screen sensing cell array in addition to the partial sensing cell array. Therefore, a covering range of the optical sensing cells 32 is smaller than or equal to a covering range of the display unit sets 20. In addition, the in-cell optical biometrics sensor 100 may further include a protection cover layer 90 disposed on the display cover layer 10, wherein the object F is placed above the sensing substrate 30, or placed on or above the protection cover layer 90.

Referring to FIGS. 1 and 2A, the in-cell optical biometrics sensor 100 further includes the optical modules 33 respectively disposed adjacently to the optical sensing cells 32 (respectively disposed on or above the optical sensing cells 32 in this embodiment). Disposing two elements adjacently to each other may indicate that no distance is present between the two elements, so that the direct connection state is present therebetween, and may also indicate that a distance is present between the two elements. In this example, the optical module 33 includes a light shielding layer 34 for shielding stray light, and the optical modules 33 and the optical sensing cells 32 form the sensing cells 31. The optical module 33 transmits a predetermined viewing angle of light, coming from the object F, to the optical sensing cell 32. The display cover layer 10 is a polarizer working in conjunction with the light of the display unit sets 20 to display information, wherein this pertains to the OLED display technology, and detailed descriptions thereof will be omitted. The sensing substrate 30 includes an OLED substrate 37 and a TFT layer 38 disposed on the OLED substrate 37. The sensing cells 31 are disposed on a partial part of the TFT layer 38 in a non-full-display sensing condition, or disposed on a full part of the TFT layer 38 in a full-display sensing condition, and the display unit sets 20 are disposed on the TFT layer 38. Actually, the TFT layer is not a single-material layer, and may even include one or multiple metal layers. Because the display panel pertains to the existing technology, detailed descriptions thereof will be omitted. The TFT layer 38 may be formed with TFTs arranged in an array. In one example, the TFT controls switching of the display unit set 20 to provide the display effect.

In FIG. 2A, the optical module 33 includes the light shielding layer 34, a micro lens 36 and a transparent dielectric layer 35. The light shielding layer 34 is disposed above the optical sensing cell 32 and has an aperture 34A disposed above the optical sensing cell 32. The micro lens 36 is disposed above the light shielding layer 34. The transparent dielectric layer 35 is disposed between the light shielding layer 34, the micro lens 36 and the optical sensing cell 32, and filled into the aperture 34A to define a focal length required by the micro lens 36. According to this structural design, the micro lens 36 can transmit the predetermined viewing angle (e.g., the divergence angle of FIG. 1) of light, coming from the object F to the optical sensing cell 32 through the transparent dielectric layer 35 and the aperture 34A, wherein other unnecessary light is regarded as stray light.

Referring to FIG. 2B, this example is similar to FIG. 2A except for the difference that the light shielding layer 34 is disposed around the optical sensing cell 32 (may include an upper portion and/or a side portion), so that the light shielding layer 34 disposed around the optical sensing cell 32 shields ambient stray light to improve the quality of the fingerprint image. The ambient stray light may come from the display unit set 20, and this is especially important when implementing in-cell optical biometrics characteristics sensing technology. It is worth noting that the light shielding layer 34 may have a one-single-layer structure or a multi-layer structure formed in the same time period or different time periods. The light shielding layer may have a two-dimensional structure (FIG. 2A) or a three-dimensional structure (FIGS. 2B and 2C).

Referring to FIG. 2C, this example is similar to FIG. 2B except for the difference that the light shielding layer 34 is also disposed around the transparent dielectric layer 35 to shield the ambient stray light, which may come from the display unit set 20, and to improve the quality of the fingerprint image.

Therefore, the key structure of this disclosure resides in that the light shielding layer 34 is formed on the lateral side and/or sides of the optical sensing cell 32 and/or the optical module 33. The light shielding layer 34 can protect the optical sensing cell 32 and/or the optical module 33 from being interfered by the lateral incident light coming from the region of the display unit set 20.

It is worth noting that in all the above-mentioned and following examples, a lower light shielding layer, which is formed by a metal layer or may be formed by any opaque layer when the optical sensing cell 32 is being formed, may further be formed under the optical sensing cell 32 to shield the stray light, coming from a location thereunder (e.g., the OLED substrate or the TFT layer), to improve the quality of the fingerprint image. Therefore, the light shielding layer disposed around the optical sensing cell 32 can avoid the top, side and/or bottom stray light interference.

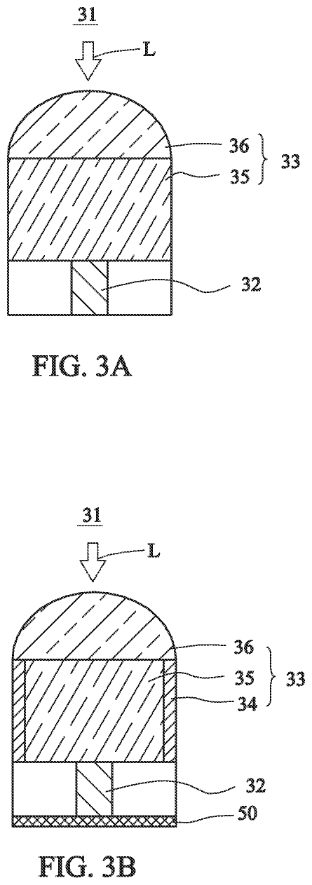

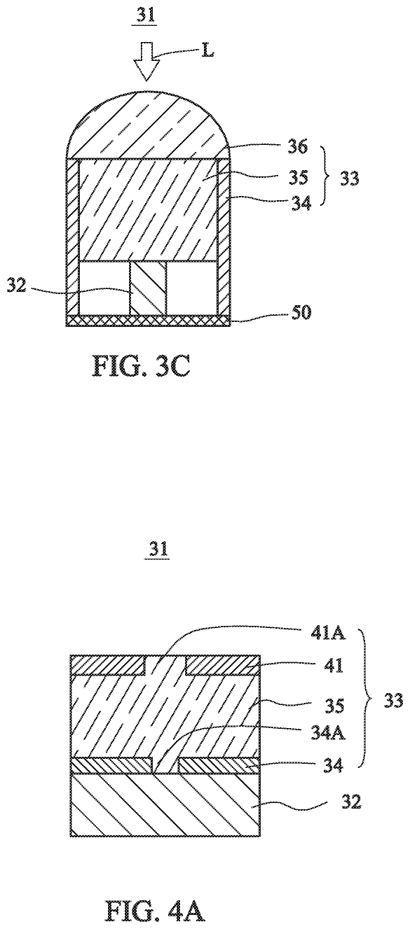

FIGS. 3A to 3C are schematically cross-sectional views showing three examples of the optical module of FIG. 1. Referring to FIG. 3A, this example is similar to FIG. 2A except for the difference that no light shielding layer is present. Therefore, the micro lens 36 of the optical module 33 is disposed above the optical sensing cell 32, and the transparent dielectric layer 35 is disposed between the micro lens 36 and the optical sensing cell 32. In this example, the light-receiving range of the optical sensing cell 32 is reduced, so that a horizontal dimension of the optical sensing cell 32 is smaller than a horizontal dimension of the micro lens 36 to provide a virtual aperture structure. With the virtual aperture structure, the micro lens 36 transmits the predetermined viewing angle of light, coming from the object F, to the optical sensing cell 32 through the transparent dielectric layer 35. Therefore, the light shielding layer may be omitted when the virtual aperture structure is provided, so that the manufacturing processes and the manufacturing costs can be reduced.

Referring to FIG. 3B, contents similar to those of FIG. 3A and detailed descriptions thereof will be omitted, wherein the difference resides in that a light shielding layer 34 is further provided. The light shielding layer 34 is disposed above the optical sensing cell 32 and around the transparent dielectric layer 35, so that the light shielding layer 34 disposed around the transparent dielectric layer 35 shields the ambient stray light to eliminate the interference caused by the adjacent optical module 33. In addition, the in-cell optical biometrics sensor 100 may further include a lower light shielding layer 50 disposed under the optical sensing cell 32. The lower light shielding layer 50 is not restricted to a single-material layer, and may also be a combination of insulating layer(s) and metal layer(s) or any opaque layer(s) as long as the bottom stray light under the optical sensing cell 32 can be shielded. The number of material layer(s) is not restricted. The insulating layer is disposed between the optical sensing cell 32 and the metal layer or opaque layer, wherein the metal layer or the opaque layer provides the light shielding effect.

Referring to FIG. 3C, contents similar to those of FIG. 3B and detailed descriptions thereof will be omitted, wherein the difference resides in that the light shielding layer 34 is disposed around the optical sensing cell 32 to shield ambient stray light of the optical sensing cell 32. In addition, the light shielding layer 34 is also disposed around the transparent dielectric layer 35 to eliminate the interference caused by the adjacent optical module 33 and display unit set 20, and the lower light shielding layer 50 can be used to eliminate the bottom stray light interference. In one example, TFTs may be firstly formed in the TFT layer or the TFT array substrate, and then the optical sensing cells 32 are formed above the TFTs, Therefore, patterns of metal wiring layer(s) of TFTs can be configured such that a portion of the metal wiring layer(s) serves as the lower light shielding layer 50, so that the metal wiring layer has both the metal wiring and light-shielding effects. It is worth noting that the lower light shielding layer 50 may also be configured and disposed under all the above-mentioned and following optical sensing cells 32.

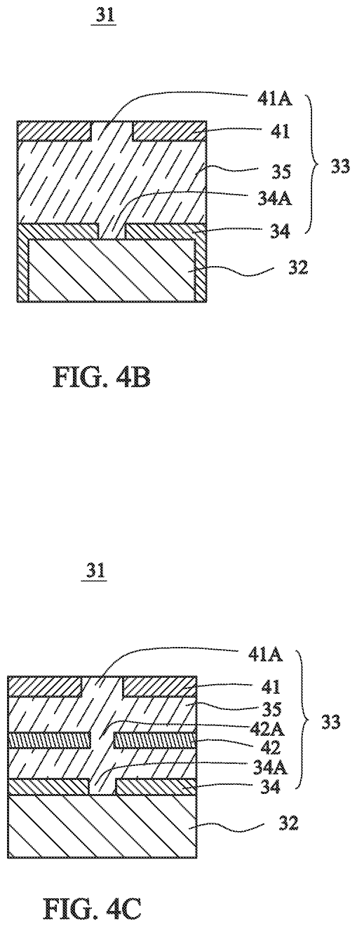

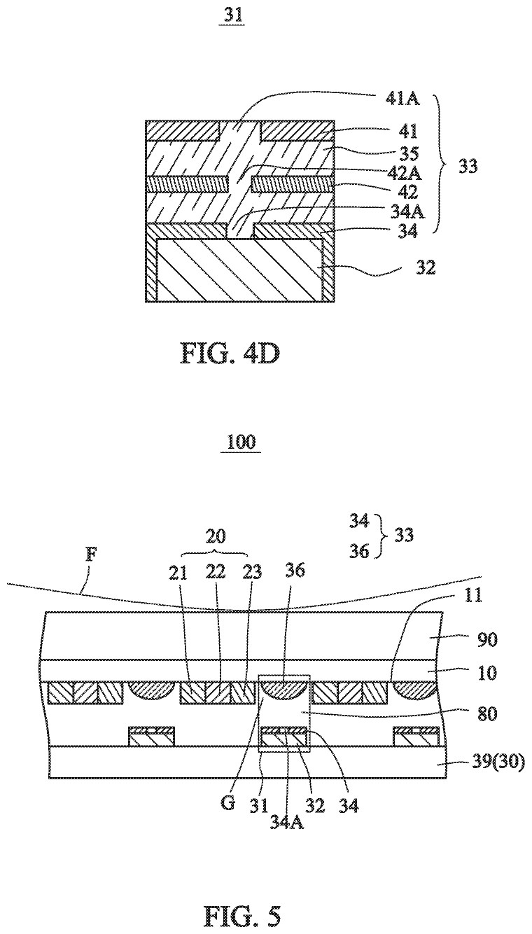

FIGS. 4A to 4D are schematically cross-sectional views showing four examples of the optical module of FIG. 1. Referring to FIG. 4A, the optical module 33 is a collimating structure, which is also a light shielding structure with multiple layers and includes a light shielding layer 34, a second light shielding layer 41 and a transparent dielectric layer 35. The light shielding layer 34 is disposed above the optical sensing cell 32, and has an aperture 34A disposed above the optical sensing cell 32. The second light shielding layer 41 is disposed above the light shielding layer 34, and has a second aperture 41A corresponding to the aperture 34A. The transparent dielectric layer 35 is disposed between the light shielding layer 34, the second light shielding layer 41 and the optical sensing cell 32, and filled into the aperture 34A and the second aperture 41A. The second aperture 41A works in conjunction with the aperture 34A to transmit the predetermined viewing angle of light, coming from the object F, to the optical sensing cell 32. In order to provide the better collimating effect, h>3(a1+a2)/2 needs to be satisfied, where h represents a distance from the light shielding layer 34 to the second light shielding layer 41, a1 represents a diameter of the aperture 34A, and a2 represents a diameter of the second aperture 41A. It is worth noting that one single aperture may correspond to one single optical sensing cell 32 or multiple apertures may correspond to one single optical sensing cell 32.

Referring to FIG. 4B, contents similar to those of FIG. 4A and detailed descriptions thereof will be omitted, wherein the difference resides in that the light shielding layer 34 is disposed around the optical sensing cell 32 to eliminate the stray light interference caused by the adjacent optical module 33 and display unit set 20.

Referring to FIG. 4C similar to FIG. 4A, the optical module 33 is a light shielding structure with multiple layers and includes a light shielding layer 34, a second light shielding layer 41, a third light shielding layer 42 and a transparent dielectric layer 35. The third light shielding layer 42 is disposed between the light shielding layer 34 and the second light shielding layer 41, and has a third aperture 42A corresponding to the second aperture 41A and the aperture 34A. The transparent dielectric layer 35 is disposed between the light shielding layer 34, the second light shielding layer 41, the third light shielding layer 42 and the optical sensing cell 32, and filled into the aperture 34A, the second aperture 41A and the third aperture 42A. The third aperture 42A works in conjunction with the second aperture 41A and the aperture 34A to transmit the predetermined viewing angle of light, coming from the object F, to the optical sensing cell 32. It is worth noting that more light shielding layers and apertures thereof may be provided to achieve the light collimating function.

Referring to FIG. 4D, contents similar to those of FIG. 4C and detailed descriptions thereof will be omitted, wherein the difference resides in that the light shielding layer 34 is disposed around the optical sensing cell 32 to eliminate the stray light interference caused by the adjacent optical module 33 and display unit set 20.

FIGS. 5 to 7 are schematically partial cross-sectional views showing three modified examples of the in-cell optical biometrics sensor of FIG. 1. Referring to FIG. 5, this example is similar to FIG. 1 except for the difference that the application pertains to the LCD occasion. Therefore, the display cover layer 10 is a filter (color filter), the optical modules 33 are respectively disposed above the optical sensing cells 32, and the optical modules 33 and the optical sensing cells 32 form sensing cells 31. The display unit set 20 and a part of the optical module 33 are disposed on a lower surface 11 of the display cover layer 10. The filter works in conjunction with the display unit set 20 to display information. The sensing substrate 30 includes a TFT array substrate 39, on which TFTs arranged in an array are formed. The optical sensing cell 32 is disposed on the TFT array substrate 39. In this example, a liquid crystal display material 80 may be filled into a gap between the display cover layer 10 and the sensing substrate 30. In addition, each optical module 33 includes a micro lens 36. The micro lens 36 has a light focusing structure, which is a concave light focusing structure with respect to the light outputted from the micro lens 36, or may also be a convex or otherwise shaped light focusing structure, such as a plasmonic light focusing structure and the like, in another embodiment. The light focusing structure focuses light onto or into the optical sensing cell 32, wherein the micro lens 36 is separated from the optical sensing cell 32. Herein, the micro lens 36 is disposed on the lower surface 11 and opposite the optical sensing cell 32 in an inverted (upside-down) state, and is different from the non-inverted micro lens of FIG. 1. However, the micro lens 36 may be formed by processes associated with those of FIG. 1. It is worth noting that the light focusing structure may be formed based on the refraction difference.

In addition, the optical module 33 may further include a light shielding layer 34 disposed on or around the optical sensing cell 32 and separated from the micro lens 36. The light shielding layer 34 has an aperture 34A, so that the optical sensing cell 32 receives light through the aperture 34A.

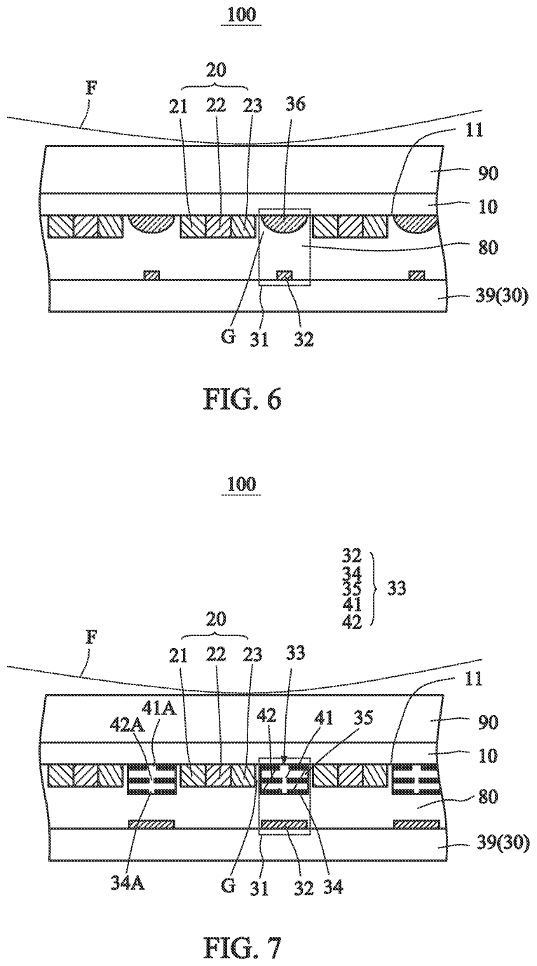

Referring to FIG. 6, the micro lens 36 is separated from the optical sensing cell 32, and the horizontal dimension of the optical sensing cell 32 is smaller than the horizontal dimension of the micro lens 36 to provide a virtual aperture structure, so that the micro lens 36 transmits the predetermined viewing angle of light, coming from the object F, to the optical sensing cell 32. Herein, the micro lens 36 also has a light focusing structure for focusing light onto the optical sensing cell 32.

Referring to FIG. 7, this example is a combination of FIGS. 5 and 4C to achieve the light collimating function similarly. In this case, the second light shielding layer 41 is disposed on the lower surface 11 of the display cover layer 10, and the light shielding layer 34 is separated from the optical sensing cell 32 by a predetermined distance. It is worth noting that one single aperture may correspond to one single optical sensing cell 32, or multiple apertures may correspond to one single optical sensing cell 32. In addition, the optical module of FIG. 4A is also applicable to FIG. 7. Therefore, each optical module 33 in FIG. 7 is a light shielding structure with multiple layers including the light shielding layer 34. The optical modules 33 and the display unit sets 20 are disposed on the lower surface 11 of the display cover layer 10, and the optical modules 33 are respectively disposed opposite the optical sensing cells 32.

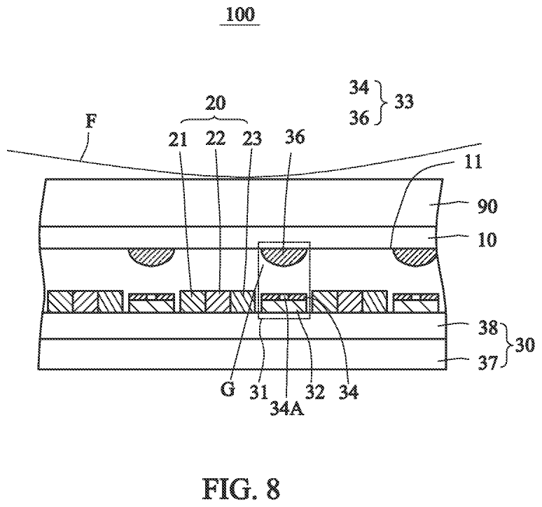

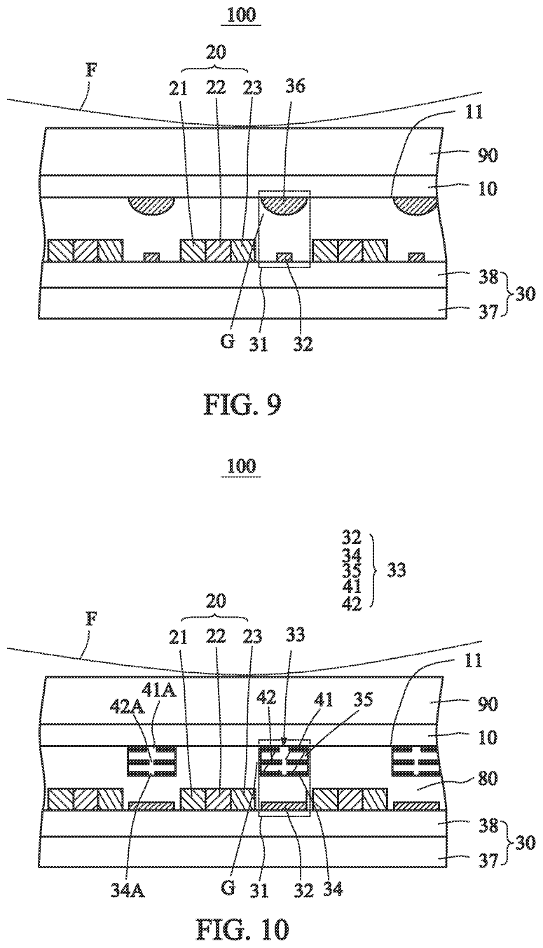

FIGS. 8 to 10 are schematically partial cross-sectional views respectively showing modified examples of the in-cell optical biometrics sensor of FIGS. 5 to 7. FIGS. 8 to 10 pertain to the OLED or it LED application, wherein the display cover layer 10 has no filter layer, but still has an optical module similar to FIGS. 5 to 7. So, the OLED or μ LED panel has a lower substrate for emitting light, and an upper substrate, on which no filter layer is provided, but the optical module is still disposed. Referring to FIG. 8, this example is similar to FIG. 5 because the micro lens 36 is disposed on the lower surface 11 of the display cover layer 10 and opposite the optical sensing cell 32, wherein the difference resides in that the display unit sets 20 are disposed on the TFT layer 38. Referring to FIGS. 9 and 10 respectively similar to FIGS. 6 and 7, the difference resides in that the display unit sets 20 are disposed on the TFT layer 38.

The light required by the optical sensing cell 32 of the in-cell optical biometrics sensor 100 may be environment light; visible light, infrared light or any other light provided by the display panel; or visible light, infrared light or any other light additionally disposed outside the display panel.

With the above-mentioned in-cell optical biometrics sensor, sensing cells for sensing biometrics characteristics may be integrated with the display panel to provide a display panel with an in-cell optical biometrics sensor, so that the display function and the biometrics characteristics sensing function can be integrated together, and that the assembling cost, and the positioning structure or bonding structure necessary for assembling can be saved. In addition, because the sensing cell can be configured in conjunction with the display pixel of the display panel, it is possible to design the in-cell optical biometrics sensor having the full-display biometrics characteristics sensing function, so that the displaying of the electronic apparatus and the biometrics characteristics sensing convenience are further enhanced.

The specific embodiments proposed in the detailed description of this disclosure are only used to facilitate the description of the technical contents of this disclosure, and do not narrowly limit this disclosure to the above-mentioned embodiments. Various changes of implementations made without departing from the spirit of this disclosure and the scope of the claims are deemed as falling within the following claims.

What is claimed is:

1. An in-cell optical biometrics sensor, comprising:
display unit sets each comprising one or multiple display units; and
optical sensing cells respectively disposed in gaps between the display unit sets; and
optical modules respectively disposed adjacently to the optical sensing cells, wherein each of the optical modules comprises a light shielding layer for shielding stray light, and the optical sensing cells sense biometrics characteristics of an object through the optical modules.

2. The in-cell optical biometrics sensor according to claim 1, wherein the optical modules are respectively disposed on the optical sensing cells, the optical modules and the optical sensing cells form sensing cells, and each of the optical modules transmits a predetermined viewing angle of light, coming from the object, to the optical sensing cell.

3. The in-cell optical biometrics sensor according to claim 2, wherein the optical sensing cells are disposed on a sensing substrate, and the sensing substrate comprises: an OLED substrate; and a TFT layer disposed on the OLED substrate, wherein the sensing cells are disposed on the TFT layer, and the display unit sets are disposed on the TFT layer.

4. The in-cell optical biometrics sensor according to claim 2, wherein each of the optical modules further comprises a micro lens, the micro lens has a light focusing structure, the optical sensing cell receives light focused by the light focusing structure, and the micro lens is disposed on a lower surface of a display cover layer and opposite the optical sensing cell.

5. The in-cell optical biometrics sensor according to claim 4, wherein the light shielding layer is disposed on or around the optical sensing cell and is separated from the micro lens, and the light shielding layer has an aperture, so that the optical sensing cell receives the light through the aperture.

6. The in-cell optical biometrics sensor according to claim 4, wherein the micro lens is separated from the optical sensing cell, a horizontal dimension of the optical sensing cell is smaller than a horizontal dimension of the micro lens to provide a virtual aperture structure, so that the micro lens transmits the predetermined viewing angle of light, coming from the object, to the optical sensing cell.

7. The in-cell optical biometrics sensor according to claim 2, wherein each of the optical modules is a light shielding structure with multiple layers comprising the light shielding layer, and the optical module is disposed on a lower surface of a display cover layer and opposite the optical sensing cell.

8. The in-cell optical biometrics sensor according to claim 1, wherein the light shielding layer is disposed above the optical sensing cell, and has an aperture disposed above the optical sensing cell, wherein each of the optical modules further comprises:
a micro lens disposed above the light shielding layer; and
a transparent dielectric layer disposed between the light shielding layer, the micro lens and the optical sensing cell, and filled into the aperture, wherein the micro lens transmits a predetermined viewing angle of light, coming from the object, to the optical sensing cell through the transparent dielectric layer and the aperture.

9. The in-cell optical biometrics sensor according to claim 1, wherein the light shielding layer is disposed around the optical sensing cell, and has an aperture disposed above the optical sensing cell, wherein the light shielding layer disposed around the optical sensing cell shields ambient stray light, and each of the optical modules further comprises:
a micro lens disposed above the light shielding layer; and
a transparent dielectric layer disposed between the light shielding layer, the micro lens and the optical sensing cell, and filled into the aperture, wherein the micro lens transmits a predetermined viewing angle of light, coming from the object, to the optical sensing cell through the transparent dielectric layer and the aperture.

10. The in-cell optical biometrics sensor according to claim 1, wherein each of the optical modules comprises:
a micro lens disposed above the optical sensing cell; and
a transparent dielectric layer disposed between the micro lens and the optical sensing cell, wherein a horizontal dimension of the optical sensing cell is smaller than a horizontal dimension of the micro lens to provide a virtual aperture structure, so that the micro lens transmits a predetermined viewing angle of light, coming from the object, to the optical sensing cell through the transparent dielectric layer.

11. The in-cell optical biometrics sensor according to claim 10, wherein the light shielding layer of each of the optical modules is disposed above or around the optical sensing cell and disposed around the transparent dielectric layer, wherein the light shielding layer disposed around the transparent dielectric layer shields ambient stray light.

12. The in-cell optical biometrics sensor according to claim 1, wherein the light shielding layer is disposed above or around the optical sensing cell, and has an aperture disposed above the optical sensing cell, wherein each of the optical modules further comprises:
a second light shielding layer, which is disposed above the light shielding layer and has a second aperture corresponding to the aperture; and
a transparent dielectric layer disposed between the light shielding layer, the second light shielding layer and the optical sensing cell, and filled into the aperture and the second aperture, wherein the second aperture works in conjunction with the aperture to transmit a predetermined viewing angle of light, coming from the object, to the optical sensing cell, and h>3(a1+a2)/2, where h represents a distance from the light shielding layer to the second light shielding layer, a1 represents a diameter of the aperture, and a2 represents a diameter of the second aperture.

13. The in-cell optical biometrics sensor according to claim 1, wherein the light shielding layer is disposed above or around the optical sensing cell, and has an aperture disposed above the optical sensing cell, wherein each of the optical modules further comprises:
a second light shielding layer being disposed above the light shielding layer and having a second aperture;
a third light shielding layer being disposed between the light shielding layer and the second light shielding layer, and having a third aperture corresponding to the second aperture and the aperture; and
a transparent dielectric layer disposed between the light shielding layer, the second light shielding layer, the third light shielding layer and the optical sensing cell, and filled into the aperture, the second aperture and the third aperture, wherein the third aperture works in conjunction with the second aperture and the aperture to transmit a predetermined viewing angle of light, coming from the object, to the optical sensing cell.

14. The in-cell optical biometrics sensor according to claim 1, wherein the optical sensing cells are disposed on a sensing substrate, the optical modules are respectively disposed above the optical sensing cells, and the optical modules and the optical sensing cells form sensing cells, wherein each of the optical modules transmits a predetermined viewing angle of light, coming from the object, to the optical sensing cell, the sensing substrate comprises a TFT array substrate, the optical sensing cell is disposed on the TFT array substrate, and the display unit sets are disposed on a lower surface of a display cover layer above the TFT array substrate.

15. The in-cell optical biometrics sensor according to claim 14, wherein each of the optical modules further comprises a micro lens, the micro lens has a light focusing structure, the optical sensing cell receives light focused by the light focusing structure, and the display unit sets and the micro lenses are disposed on the lower surface of the display cover layer.

16. The in-cell optical biometrics sensor according to claim 15, wherein the light shielding layer is disposed on or around the optical sensing cell and is separated from the micro lens, and the light shielding layer has an aperture, so that the optical sensing cell receives the light through the aperture.

17. The in-cell optical biometrics sensor according to claim 15, wherein the micro lens is separated from the optical sensing cell, a horizontal dimension of the optical sensing cell is smaller than a horizontal dimension of the micro lens to provide a virtual aperture structure, so that the micro lens transmits the predetermined viewing angle of light, coming from the object, to the optical sensing cell.

18. The in-cell optical biometrics sensor according to claim 14, wherein each of the optical modules is a light shielding structure with multiple layers comprising the light shielding layer, and the optical modules and the display unit sets are disposed on the lower surface of the display cover layer.

19. The in-cell optical biometrics sensor according to claim 1, further comprising a lower light shielding layer disposed under each of the optical sensing cells to shield stray light coming from a location under each of the optical sensing cells.
